(19) United States
(12) Patent Application Publication
(10) Pub. No.: US 2024/0033274 A1
(43) Pub. Date: Feb. 1, 2024

(54) USE OF CANNABIS TO MITIGATE EFFECTS OF CHEMICAL EXPOSURE

(71) Applicant: Stratos IP, Inc., Pueblo, CO (US)

(72) Inventors: Jason Neely, Colorado Springs, CO (US); Brenda Verghese, Colorado Springs, CO (US)

(21) Appl. No.: 18/359,686

(22) Filed: Jul. 26, 2023

Related U.S. Application Data
(60) Provisional application No. 63/392,547, filed on Jul. 27, 2022.

(51) Int. Cl. A61K 31/00; A61K 36/185

(52) U.S. Cl. CPC A61K 31/658; A61K 36/185

(57) ABSTRACT

The use of cannabis and/or its parts (e.g., cannabinoids, CBD, THC, cannabidiol, hemp) to treat or reduce the effects caused by exposure to glyphosate, glyphosate derivatives, glyphosate disorders, and/or glyphosate toxicity in the human body and/or other animals. Also, methods of administering, to a patient (either a human or an animal), a product derived from the cannabis plant to treat exposure to glyphosate, glyphosate toxicity and/or glyphosate disorder.

### CROSS-REFERENCE

This application claims priority under 35 U.S.C. 119(e) to U.S. provisional application Ser. No. 63/392,547 filed Jul. 27, 2022 and titled USE OF CANNABIS TO MITIGATE EFFECTS OF GLYPHOSATE EXPOSURE, the entire disclosure of which is incorporated herein by reference for all purposes.

### BACKGROUND

Glyphosate is a broad-spectrum systemic herbicide and crop desiccant. It is an organophosphorus compound, specifically a phosphonate, which acts by inhibiting the plant enzyme 5-enolpyruvylshikimate-3-phosphate synthase. It is used to kill weeds, especially annual broadleaf weeds and grasses that compete with crops. Glyphosate is the most commonly used weed and grass killer and is used commercially and by private homeowners.

Products containing glyphosate are sold in various forms, including as liquid concentrate, solid, and ready-to-use liquid, for agricultural, commercial, and residential uses. Glyphosate can be applied using a wide range of application methods, including aerial sprays, ground broadcast sprayers of various types, shielded and hooded sprayers, wiper applicators, sponge bars, injection systems, and controlled droplet applicators.

There is controversy whether or not glyphosate causes adverse effects on humans and animals for those who come into contact with glyphosate. The likelihood of glyphosate exposure exists for pesticide applicators; however, the Environment Protection Agency (EPA) has concluded there is low potential for the general public or for non-applicators to be exposed to a recurring biologically relevant dose of glyphosate based on models of glyphosate persistence in the environment and dietary exposure. Many disagree.

For example, over the years, as the use of glyphosate on wheat has increased, the occurrence of celiac disease has similarly increased. Similarly, as the use of glyphosate overall has increased, the occurrence of dementia has similarly increased. However, the increased diagnosis of celiac disease may be due to it being more recognized by medial practitioners, and the increase in dementia may be due to increased longevity.

Nevertheless, if glyphosate does detrimentally affect human and animal health, it would be beneficial to reduce the effects.

### SUMMARY

The present disclosure provides for the treatment of symptoms and reduction of the effects caused by exposure to glyphosate and other certain chemicals with cannabis or parts thereof. In some instances, treatment with cannabis inhibits or mitigates glyphosate toxicity.

In one particular implementation, this disclosure provides a method comprising administering to a patient at least 0.1 mg cannabinoids per day for the treatment of symptoms from glyphosate exposure. The treatment may be, e.g., at least 30 days or at least 90 days.

In another particular implementation, this disclosure provides a method comprising treating a patient with at least 0.1 mg cannabinoids per day for symptoms from glyphosate exposure.

In yet another particular implementation, this disclosure provides a method comprising administering to a patient at least 0.1 mg cannabinoids per day for the inhibition of glyphosate toxicity.

These and other aspects of the invention described herein will be apparent after consideration of the Detailed Description and Figures herein. It is to be understood, however, that the scope of the claimed subject matter shall be determined by the claims as issued and not by whether given subject matter addresses any or all issues noted in the Background or includes any features or aspects recited in the Summary.

### BRIEF DESCRIPTION OF THE FIGURES

FIG. 1 shows the chemical structure of glyphosate.

FIG. 2 is a schematic diagram showing the mechanism of molecular transmission for a presynaptic terminal and postsynaptic terminal.

### DETAILED DESCRIPTION

As indicated above, the present disclosure is directed to the use of cannabis and/or its parts (e.g., cannabinoids, CBD, THC, cannabidiol, hemp) to treat or reduce the effects caused by exposure to glyphosate or glyphosate derivatives, glyphosate disorders and the symptoms thereof, and/or glyphosate toxicity in the human body and/or other animals. The present disclosure also is directed to methods of administering, to a patient (either a human or an animal), a product derived from the cannabis plant to treat exposure to glyphosate, glyphosate toxicity and/or glyphosate disorder. The present disclosure is also directed to methods of inhibiting and mitigating glyphosate toxicity.

Cannabis, also known as marijuana, refers to a group of three plants with psychoactive properties, known as Cannabis sativa, Cannabis indica, and Cannabis ruderalis. Native to Central and South Asia, cannabis has been used as a drug for both recreational and entheogenic purposes and in various traditional medicines for centuries. Tetrahydrocannabinol (THC) is the main psychoactive component of cannabis, which is one of 483 known compounds in the plant, including at least 65 cannabinoids, one of which is cannabidiol (CBD).

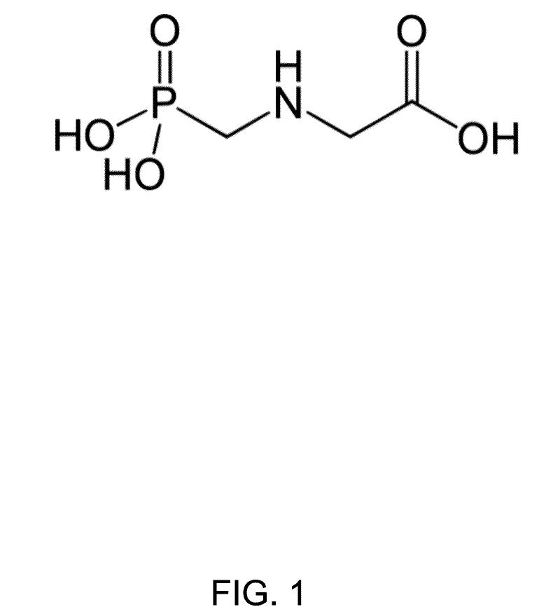

Glyphosate, also referred to as N-(phosphonomethyl)glycine, is a broad-spectrum herbicide, also called a weed killer, typically directed toward most broadleaf plants. See FIG. 1 for the chemical structure of glyphosate. Glyphosate works by inhibiting the action of a plant enzyme that plays an important role in the synthesis of phenylalanine, tyrosine, and tryptophan, three plant amino acids.

Glyphosate is important in the production of fruits, vegetables, nuts, and glyphosate-resistant field crops such as corn and soybean. It is effective at managing invasive and noxious weeds. In addition, glyphosate breaks down in the environment, can be used for no-till and low-till farming which can reduce soil erosion, reduce tractor fuel usage, and increase yield, and is also useful for integrated pest management.

Agricultural uses include corn, cotton, canola, soybean, sugar beet, alfalfa, berry crops, brassica vegetables, bulb vegetables, fruiting vegetables, leafy vegetables, legume vegetables, cucurbit vegetables, root tuber vegetables, cereal grains, grain sorghum, citrus crops, fallow, herbs and spices, orchards, tropical and subtropical fruits, stone fruits, pome fruits, nuts, vine crops, oilseed crops, and sugarcane. Nonagricultural uses include conservation land, pastures, rangeland, aquatic areas, forests, turf grass, residential areas, non-food tree crops (e.g., pine, poplar, Christmas trees), rights of way, commercial areas, paved areas, spot treatments, ornamentals, parks, and wildlife management areas.

The following description provides specific implementations. It is to be understood that other implementations are contemplated and may be made without departing from the scope or spirit of the present disclosure. The following detailed description, therefore, is not to be taken in a limiting sense. While the present disclosure is not so limited, an appreciation of various aspects of the disclosure will be gained through the discussion and the examples, provided below.

As discussed above, contact with glyphosate may cause adverse effects on human and animal health, Immediate symptoms to exposure to glyphosate are irritation in the nose and/or throat, with possible increased saliva. Other symptoms include burns in the mouth and throat, nausea, loss of appetite, vomiting, and diarrhea. The effects of long term exposure to glyphosate include dysbiosis or “leaky gut,” gut anxiety, neurotoxicity, the substitution of glyphosate for glycine, and even adverse reactions to some vaccines. Many of these symptoms fall within a broad category referred to herein as gastrointestinal symptoms or conditions, these including nausea, loss of appetite, vomiting, diarrhea, dysbiosis or “leaky gut,” gut anxiety, irritable bowel syndrome (IBS), acid reflux, and flatulence.

Although the exact mechanism is unclear, glyphosate breaks down to aminomethylphosphonic acid (AMPA) and acetic acid. At a physiological pH, acetic acid converts to acetate over time, leading to an increase in acetate.

In addition to acetate playing a playing a critical role in many cellular pathways, increased acetate leads to increased adenosine, which is one of the four nucleoside building blocks of RNA and its derivative deoxyadenosine is a building block of DNA.

Increased adenosine leads to increased serotonin, which can cause the symptoms and adverse effects described above. As adenosine converts or breaks down, other adverse effects occur.

Other derivatives of adenosine include the energy carriers adenosine mono-, di-, and triphosphate, also known as AMP/ADP/ATP. Cyclic adenosine monophosphate (cAMP) is another derivative and is pervasive in cell signal transduction, being paramount in cellular pathways. When combined with protein kinase A (PKA), the cAMP/PKA pathways play a role in the activity of serotonin in adrenocortical cells. The activation of the cAMP/PKA pathway is associated with calcium influx through T-type calcium channels.

This conversion of adenosine to cAMP (cyclic adenosine monophosphate) may be quick in the body, and can cause increased and uncontrolled signaling of neurons. This uncontrolled signaling can cause the symptoms such as burning in the mouth and throat, nausea, loss of appetite, vomiting, diarrhea, dysbiosis or “leaky gut,” and gut anxiety.

One of the presumed reasons for these and other symptoms, in addition to the increase of cAMP, is due to glyphosate's effect on the calcium channels within cells. Via the calcium channels, adenosine increases the transport, across the blood-brain-barrier, of amyloid plaque antibodies, which have been shown to increase the occurrence of Parkinson's disease, Alzheimer's disease, Multiple Sclerosis (MS), and cancers of the central nervous system.

Via such mechanisms, glyphosate increases acetic acid and thus acetate, which increases adenosine, which converts to cAMP leading to increased and uncontrolled signaling of neurons, resulting in the symptoms and adverse effect attributed to glyphosate. Adenosine also increases serotonin, which can cause the symptoms and adverse effects, and also affects the calcium channels.

Other drugs or chemicals that result in production of cAMP, like glyphosate, include lithium, mGluR antagonists, and PDE-4 inhibitors; these chemicals have a common action of up-regulating cAMP signaling. The symptoms and adverse effects caused by these chemicals are similar to those caused by glyphosate exposure, particularly the gastrointestinal symptoms. For example, in mild lithium toxicity, common symptoms include weakness, worsening tremors, mild ataxia, poor concentration, and diarrhea. With increased lithium toxicity, vomiting, the development of a gross tremor, slurred speech, confusion and lethargy emerge. mGlur antagonists are used for treating schizophrenia, anxiety, depression, and behavioral effects of drug abuse, although some patients have adverse side effects including nausea and diarrhea. PDE-4 inhibitors are used for the treatment of inflammatory airway diseases, psoriatic arthritis, and atopic dermatitis. However, the side effects of the drugs include nausea, emesis, and gastrointestinal reactions.

It has been found that administering cannabis and/or its parts (e.g., cannabinoids, CBD, THC, hemp) to a patient (e.g., human, animal) experiencing symptoms of long term exposure to glyphosate or other chemicals such as lithium, mGluR antagonists, and PDE-4 inhibitors, can mitigate these symptoms and adverse effects, for example, by decreasing the calcium influx into presynaptic neurons thereby causing unwanted firing/signaling. The activation of the CB1 and CB2 receptors reduces the Ca+2 influx into the presynaptic neuron thereby preventing neurotransmitter release and signal activation.

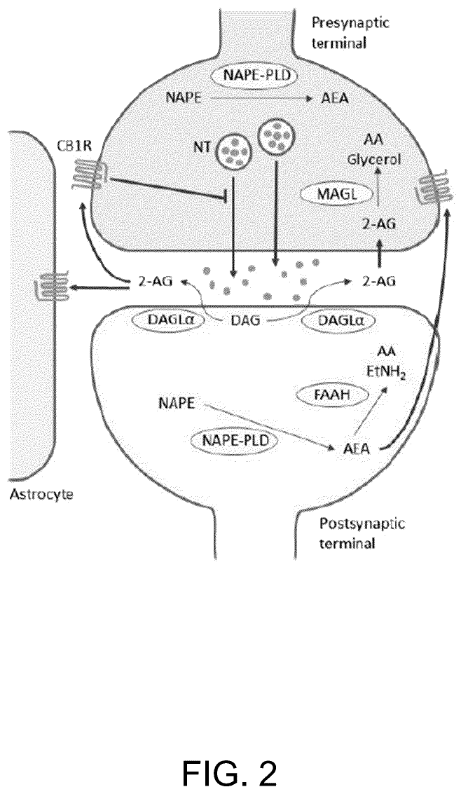

Cannabinoids, including cannabidiol, act as an inverse agonist to inhibit the signaling that occurs through a reduced number of pathways. FIG. 2 shows a simplified schematic diagram of endocannabinoid retrograde signaling mediated synaptic transmission, the endocannabinoids being lipid molecules similar to cannabinoids, but naturally produced by the body.

Endocannabinoids are produced from postsynaptic terminals upon neuronal activation. The two major endocannabinoids shown in FIG. 2 are 2-acrachidonalyglycerol (2-AG), which is biosynthesized from diacylglycerol (DAG) by diacylglycerol lipase-α (DAGα), and anandaminde (AEA), which is synthesized from N-acyl-phosphatidylethanolamine (NAPDE) by NAPE-specific phospholipase D (NAPE-PLD). Endocannabinoids, mainly 2-AG, readily cross the membrane and travel in a retrograde fashion to active CBlRs located in presynaptic terminals. Activated CBlRs will then inhibit neurotransmitter (NT) release through the suppression of calcium influx. 2-AG is also able to activate CBlRs located in astrocytes, leading to the release of glutamate. Extra 2-AG in the synaptic cleft is taken up into the presynaptic terminals, via a yet unclear mechanism, and degraded to arachidonic acid (AA) and glycerol by monoacylglycerol lipase (MAGL). On the other hand, AEA, synthesized in postsynaptic terminal, activates intracellular CB1R and other non-CBR targets, such as the transient receptor potential cation channel subfamily V member 1 (TRP1). Although endocannabinoid retrograde signaling is mainly mediated by 2-AG, AEA can activate presynaptic CB1Rs as well. Fatty acid amide hydrolase (FASH) is primarily found in postsynaptic terminals and is responsible for degrading AEA to AA and ethanolamine (EtNH2). Although NAPE-PLD is expressed in presynaptic terminals in several brain regions, it is not clear yet whether AEA is responsible for anterograde signaling in the endocannabinoid systems. Note that alternative routes exist for the metabolism of endocannabinoids, depending on the brain regions and physiological conditions. In FIG. 2, thin arrows indicated enzymatic processes, thick arrows indicate translocation, and the blunted arrow indicates inhibition.

Based on this, because the endocannabinoids suppress calcium influx, it is believed that cannabinoids, similarly, decrease the calcium channels that allow the glyphosate or other chemical affecting calcium transport to affect cells. Thus, administering cannabinoids to a patient (e.g., human, animal) experiencing symptoms of exposure to glyphosate or other chemical can mitigate the symptoms and the effects and can inhibit future symptoms and effects.

Because people vary from being extensive metabolizers to poor metabolizers, the amount of cannabinoid receptors differs for each individual. Because of this, the optimal dose will differ for each individual, however, a lowest dose (for most individuals) is 1 mg of cannabinoids once a day (QD) and a highest dose (for most individuals) is 1000 mg of cannabinoids a day, e.g., administered as 250 mg of cannabinoids four times a day (QID). For some individuals, for some purposes, a dose of 0.1 mg of cannabinoids is effective. An average dose, for most individuals, is 20 mg of cannabinoids a day, e.g., administered as 10 mg of cannabinoids twice a day (BID). Examples of other suitable doses are 25 mg of cannabinoids a day, administered once a day (QD); 30 mg of cannabinoids a day, administered as 10 mg three times a day (TID); 90 mg of cannabinoids a day, administered as 30 mg three times a day (TID); 100 mg of cannabinoids a day, administered as 25 mg four times a day (QID). The treatment may be administered daily for at least, e.g., 5 days, 7 days, 10 days, 30 days, 90 days, or 120 days. In some instances, particularly for high level and/or long term exposures, longer treatments may be desired; for example, for most instances at least 30 days or even at least 90 days of treatment is desired.

Although the doses above are provided for cannabinoids, for the treatment of exposure to glyphosate, glyphosate disorder, glyphosate toxicity, and symptoms (e.g., gastrointestinal symptoms) due to exposure to other chemicals, any part of the cannabis plant (e.g., CBD, THF, etc.) may be administered to a patient.

For some patients and/or for some symptoms, full spectrum cannabis oil, most preferably CBD oil, is better than isolate; this may be due to the phenomenon called the entourage effect, which is the mechanism by which cannabis compounds act synergistically to modulate overall benefits and effects of the plant. The body is able to absorb and/or metabolize cannabinoids better when other parts of the cannabis plant (e.g., cannabis sativa plant), parts such as fatty acids, lipids, terpenes, and other extracted components, are present with the cannabinoids. One particular mix of cannabinoids is THC and CBD (e.g., in a 50/50 ratio). Other ratios and other mixes of cannabinoids can be used.

The cannabinoids can be obtained from the cannabis plant by any suitable method. The resulting extract may contain, in addition to the cannabinoids, fatty acids, fats, lipids, and/or terpenes. The cannabinoids may be extracted from any variety of cannabis.

The extract is prepared from cannabis flowers by any of the following methods and combination, but not limited to, water extraction and pressure (solvent less), carbon dioxide, hydrocarbon butane and/or propane, alcohol (e.g., ethanol).

First, flowers produced by the cannabis plant are harvested. These flowers can be immediately processed for their extract or can be dried or frozen for future use.

To begin the extraction processes, the flowers may be pressed, chopped, or ground. The extraction may be: water or solventless extraction, either as water hash or rosin (which is water hash with pressure and heat); a hydrocarbon extraction, either as live resin (which uses frozen flowers extracted with a hydrocarbon), shatter, budder, or wax; a supercritical extraction, with, e.g., carbon dioxide or supercritical carbon dioxide; or an alcohol extraction, e.g., with ethanol. The resulting extract may be decarboxylated.

The resulting extract can be ingested in this form or can be further processed, e.g., decarboxylated to create a cannabinoid decarboxylated extract. The extract and/or decarboxylated extract is mixed with other ingredients to make a more preferable dosage form for ingestion.

Detailed examples are provided below:

Example #E1: The cannabis plant (e.g., flowers or leaves) and water (e.g., cold water) are mixed together to separate the plant material from the trichome. The trichomes are collected by removing (e.g., straining, filtering, decanting) the water. The trichomes are then pressed and/or heated creating a cannabis extract.

Example #E2: A hydrocarbon such as propane or butane is passed through the cannabis (e.g., flowers or leaves). The hydrocarbon is removed by various methods, producing a cannabis extract. One particular hydrocarbon removal method includes butane and/or propane.

Example #E3: Carbon Dioxide is passed through the cannabis plant (e.g., flowers or leaves). The carbon dioxide is removed, producing a cannabis extract. In a particular example, supercritical carbon dioxide is passed through a chamber containing the cannabis and pumped to another chamber where the supercritical carbon dioxide is removed, thus resulting in a cannabis extract.

Example #E4: An alcohol, e.g., ethanol, is passed through the cannabis plant (e.g., flowers or leaves), thus steeping the cannabinoids from the plant. The alcohol is removed (e.g., strained, filtered, decanted) producing a cannabis extract.

Example #E5: Rather than producing an extract from the cannabis, parts of the cannabis plant itself, may be used. For example, any or all of the flowers, leaves, and stems are ground, chopped, or powdered.

Example #E6: The cannabis extract from any of Examples E1-E5 can be decarboxylated using heat. A vacuum may be used in addition to heat to perform the decarboxylation step at a lower temperature.

The extract may be incorporated into an edible product, e.g., a tablet, capsule, gelatin-type edible, gummy-type edible, hard candy-type edible, etc. In some embodiments, the cannabis plant, itself, can be incorporated into an edible product. Various examples for producing an edible product are provided below:

Example #P1: Capsule. The cannabis plant is ground up and filled into a swallowable capsule.

Example #P2: Syringe. The extract and/or decarboxylated extract, as a liquid, is administered orally, e.g., via syringe, or dropper.

Example #P3: Capsule. The extract and/or decarboxylated extract is filled into a swallowable capsule.

Example #P4: Tablet. The decarboxylated extract and a thickener, such as hydroxypropyl cellulose, are dissolved in a solvent, e.g., ethanol. This mixture is mixed with microcrystalline cellulose, starch, and sodium starch glycolate and dried to remove the solvent. Magnesium stearate and colloidal silicon dioxide is added to the dried mixture. The resulting mixture is compressed into tablets.

Example #P5: Tincture. The extract is heated to decarboxylate the extract producing a decarboxylated extract. The decarboxylated extract is then infused into a base solution of a medium triglyceride oil and flavorings. The solution is heated, blended and homogenized and used as an oral solution.

The extract may be incorporated into a transdermal compound, to be applied to and absorbed directly into the skin. The entire product may absorb into the skin, or the product may include a carrier, such as a patch. In some embodiments, the cannabis plant, itself, can be incorporated into a transdermal product. An example for producing a transdermal product is provided below:

The extract is heated to decarboxylate the extract, producing a decarboxylated extract. The decarboxylated extract is dissolved into a base solution, e.g., coconut oil and/or shea butter and a penetration modifier such dimethyl isosorbide (DMI) or diethylene glycol monoethyl ether (DGME). The blend is heated, homogenized, and designated for topical use.

The extract or cannabis plant itself may be incorporated into other product forms for administration to a patient, products such as vaporizable forms, tinctures, oils, sprays, salves and lotions, teas, and smokable forms.

The product, whether an edible, transdermal, or other, has a concentration of cannabinoid therein to provide an affective amount of cannabinoid to the patient. In some embodiments, each product has at least 0.1 mg of cannabinoid, in other embodiments at least 1 mg of cannabinoid, in other embodiments at least 5 mg of cannabinoid and yet in other embodiments at least 10 mg cannabinoid, so that the products provide a dosage of at least 0.1 mg, at east 1 mg, at least 5 mg, or at least 10 mg cannabinoid to the patient.

To effectively counteract or treat the symptoms of glyphosate or other chemical exposure, a lowest dose (for most individuals) is 1 mg of cannabinoids once a day (QD) and a highest dose (for most individuals) is 1000 mg of cannabinoids a day, e.g., administered as 250 mg of cannabinoids four times a day (QID). An average dose, for most individuals, is 20 mg of cannabinoids a day, e.g., administered as 10 mg of cannabinoids twice a day (BID). Other dosages are possible, examples of which are provided above. One or multiple products may be ingested by or otherwise administered to the patient to obtain the total dosage.

The administered cannabinoids, at a proper dosage, are effective at reducing symptoms of various diseases and conditions associated with exposure to glyphosate or other chemicals having similar symptoms. For example, dysbiosis or “leaky gut” has many more symptoms than just digestive issues (e.g., gas, bloating, diarrhea, irritable bowel syndrome (IBS)), including food allergies or food intolerances, seasonal allergies, brain fog, mood imbalances, hormonal imbalances, skin issues, autoimmune diseases such as rheumatoid arthritis, lupus, psoriasis, or celiac disease, fibromyalgia, and chronic fatigue. By administering cannabinoids, at a level of at least 1 mg per day to a patient, for some patients at least 10 mg per day, and for other patients at least 20 mg per day, these symptoms can be reduced and, for some, eliminated. The cannabinoids inhibit the signaling that occurs in the presynaptic and postsynaptic terminals. See, e.g., FIG. 2 and the discussion above.

For some patients, combining the cannabinoids with another product may increase the rate or symptom reduction and/or further decrease the severity of the symptoms. These additional products may be, e.g., pectin (e.g., citrus pectin), alginates (e.g., kelp), sources of glycine, gingko biloba, and probiotics and prebiotics.

The above specification and examples provide a complete description of the structure and use of exemplary implementations of the invention, particularly, of the use of Cannabis sativa (cannabinoids, CBD, THC, hemp, cannabis) to treat or reduce the effects of glyphosate/glyphosate toxicity and other chemicals producing the same symptoms.

The above description provides specific implementations. It is to be understood that other implementations are contemplated and may be made without departing from the scope or spirit of the present disclosure. The above detailed description, therefore, is not to be taken in a limiting sense. While the present disclosure is not so limited, an appreciation of various aspects of the disclosure will be gained through a discussion of the examples provided.

Unless otherwise indicated, all numbers expressing feature sizes, amounts, and physical properties are to be understood as being modified by the term “about,” whether or not the term “about” is immediately present. Accordingly, unless indicated to the contrary, the numerical parameters set forth are approximations that can vary depending upon the desired properties sought to be obtained by those skilled in the art utilizing the teachings disclosed herein.

As used herein, the singular forms “a”, “an”, and “the” encompass implementations having plural referents, unless the content clearly dictates otherwise. As used in this specification and the appended claims, the term “or” is generally employed in its sense including “and/or” unless the content clearly dictates otherwise.

What is claimed is:

1. A method comprising administering to a patient at least 0.1 mg cannabinoids per day for at least 30 days for the treatment of symptoms from glyphosate exposure.

2. The method of claim 1, comprising administering to a patient at least 10 mg cannabinoids per day.

3. The method of claim 1, comprising administering to a patient at least 20 mg cannabinoids per day.

4. The method of claim 3, comprising administering to a patient at least 20 mg cannabinoids per day at a dosage of 10 mg twice a day.

5. The method of claim 1, wherein the symptoms from glyphosate exposure are one or more of gastrointestinal symptoms, neurotoxicity, and adverse reactions to some vaccines.

6. The method of claim 1, wherein the treatment of symptoms includes a reduction in severity of the symptoms and/or an increase in rate of reduction of the symptoms.

7. The method of claim 1, wherein the cannabinoids comprise one or both of THC and CBD.

8. A method comprising treating a patient with at least 0.1 mg cannabinoids per day for at least 30 days for symptoms from glyphosate exposure.

9. The method of claim 8, wherein the symptoms are gastrointestinal symptoms.

10. The method of claim 8, comprising treating the patient with at least 10 mg cannabinoids per day.

11. The method of claim 8, comprising treating the patient with at least 20 mg cannabinoids per day.

12. The method of claim 11, comprising treating the patient with at least 20 mg cannabinoids per day at a dosage of 10 mg twice a day.

13. The method of claim 8, wherein treating the patient includes a reduction in severity of the symptoms and/or an increase in rate of reduction of the symptoms.

14. The method of claim 8, wherein the cannabinoids comprise one or both of THC and CBD.

15. A method comprising treating a patient with at least 0.1 mg cannabinoids per day for at least 5 days for gastrointestinal symptoms caused by chemical exposure.

16. The method of claim 15, comprising treating the patient with at least 10 mg cannabinoids per day.

17. The method of claim 15, comprising treating the patient with at least 20 mg cannabinoids per day.

18. The method of claim 17, comprising treating the patient with at least 20 mg cannabinoids per day at a dosage of 10 mg twice a day.

19. The method of claim 15, wherein the cannabinoids comprise one or both of THC and CBD.
